(19) United States
(12) Patent Application Publication
(10) Pub. No.: US 2026/0176269 A1
(43) Pub. Date: Jun. 25, 2026

(54) ANTICOAGULANT HEPARIN OLIGOSACCHARIDE PHENYL-LINKED DIMER, PREPARATION METHOD THEREFOR AND USE THEREOF

(71) Applicant: SHANDONG UNIVERSTIY, Jinan (CN)

(72) Inventors: Chunhui LIU, Jinan (CN); Guijiao ZHANG, Jinan (CN); Yaqi QIU, Jinan (CN); Jingru LI, Jinan (CN); Kaihua YANG, Jinan (CN); Yanzhen CHENG, Jinan (CN)

(73) Assignee: SHANDONG UNIVERSTIY, Jinan (CN)

(21) Appl. No.: 19/125,775

(22) PCT Filed: Aug. 8, 2023
(86) PCT No.: PCT/CN2023/111635

§ 371 Date: Apr. 30, 2025

(30) Foreign Application Priority Data
Sep. 1, 2022 (CN) 202211066891.1

(51) Int. Cl. C07D 471/04; A61K 9/00; A61K 31/4196; A61K 31/727; A61K 38/00

(52) U.S. Cl. CPC C07D 471/04; A61K 9/0019; A61K 31/4196; A61K 31/727; A61K 38/00

(57) ABSTRACT

An anticoagulant phenyl-linked heparin oligosaccharide dimer, a preparation method therefor and use thereof are provided. The anticoagulant phenyl-linked heparin oligosaccharide dimer has a structure as represented by general formula I, and the phenyl may be replaced with substituted phenyl, heteroaryl or substituted heteroaryl. The described phenyl-linked heparin oligosaccharide dimer may be synthesized in fewer steps, has a significantly higher total yield, potent and specific antithrombin-dependent anti-Factor Xa activity without significant anti-FactorIIa activity, the anticoagulant activity may be efficiently neutralized by protamine sulfate, has excellent pharmacokinetic profiles, making it suitable for the preparation of novel anticoagulant and antithrombotic drugs that are cost-effective, higher-quality and safer.

### TECHNICAL FIELD

The present disclosure relates to an anticoagulant phenyl-linked heparin oligosaccharide dimer, a preparation method therefor and use thereof, and belongs to the technical field of biomedicine.

### BACKGROUND TECHNOLOGY

Heparin has been known as a natural polysaccharide anticoagulant for a long time, and is still widely used in clinical indications such as thromboembolic diseases, myocardial infarction, cardiovascular surgery, cardiac catheterization, extracorporeal circulation, and hemodialysis. The global market for heparin has reached $8 billion and is showing a continuous growth trend. Currently, commercialized natural heparin drugs mainly include unfractionated heparin (UFH) extracted from pig small intestinal mucosa and different low-molecular-weight heparins (LMWH) thereof prepared by chemical or enzymatic partial depolymerization. LMWH has gradually surpassed UFH as the preferred clinical anticoagulant due to its long half-life in vivo, high bioavailability, low side effects and safe use, but its anticoagulant activity is generally lower than that of UFH. Both UFH and LMWH from animal tissue sources face challenges such as impurity contamination and a fragile supply chain of raw materials. Additionally, as a family of highly sulfated glycosaminoglycans, they have variable sulfation, isomerization, and other modifications are unevenly distributed along the saccharide chain, forming a highly complex and in homogeneous fine structure. This leads to insurmountable clinical limitations.

It has been confirmed by studies that the common structural basis for UFH and LMWH to exert anticoagulant effect is the unique pentasaccharide sequence (abbreviated as: GlcNS/Ac6S-GlcA-GlcN6S3S-IdoA2S-GlcNS6S) in heparin chain that specifically binds to antithrombin (AT). The methyl glycoside derivative of this sequence, Fondaparinux (Arixtra®), was successfully launched in 2001, and is currently the only fully synthetic heparin-like compound approved for clinical use. However, despite Fondaparinux has excellent clinical anticoagulation effect, its anti-Xa activity can't be neutralized by protamine sulfate as with animal-derived heparins, which limits the ability to terminate the anticoagulation treatment according to the treatment process, and effectively avoid the adverse reaction such as bleeding, leading to greatly limited clinical application thereof. Therefore, it is of great clinical value to develop new anticoagulant heparin molecules that is capable of being neutralized by protamine sulfate.

Although protamine sulfate has a long history as a reversal agent for animal-derived heparin in clinical practice, research thereon is not systematic and in-depth. It is generally believed that the chain length, charge density and distribution of heparin jointly determine the neutralization efficiency by protamine sulfate. For example, Professor Jian Liu in the United States synthesized a heparin dodecasaccharide containing a single AT-binding pentasaccharide and four consecutive bisaccharides trisulfate (IdoA2S-GlcNS6S) by a chemoenzymatic method, this heparin dodecasaccharide exhibited anticoagulant activity that can be effectively neutralized by protamine sulfate, and is widely regarded as the smallest heparin molecule that can be neutralized by protamine sulfate. However, it requires 22-23 steps for synthesizing the above-mentioned neutralizable anticoagulant heparin dodecasaccharide, which is complicated, costly and low in total yield. Moreover, the heparin dodecasaccharide is characterized by a short in vivo half-life, along with potential adverse reactions such as heparin-induced thrombocytopenia (HIT).

### CONTENT OF THE INVENTION

Aiming at the shortcomings of the prior art, the present disclosure provides an anticoagulant phenyl-linked heparin oligosaccharide dimer, preparation method therefor and use thereof.

### Abbreviations

AT: antithrombin
UDP-GlcNTFA: uridine diphosphate-N-trifluoroacetyl glucosamine
UDP-GlcNAc: uridine diphosphate-N-acetylglucosamine
UDP-GlcA: uridine diphosphate-glucuronic acid
PAPS: 3′-phosphoadenosine-5′-phosphosulfate
KfiA: Escherichia coli K5 N-acetylglucosamine transferase
PmHS2: Pasteurella multocida heparosan synthase 2
NST: N-sulfate transferase
C₅-epi: C₅-epimerase
2OST: 2-O-sulfotransferase
6OST: 6-O-sulfotransferase
3OST: 3-O-sulfotransferase

The technical solutions of the disclosure are as follows:

A first objective of the present disclosure is to provide an anticoagulant phenyl-linked heparin oligosaccharide dimer.

The anticoagulant phenyl-linked heparin oligosaccharide dimer or a pharmaceutically acceptable salt thereof, having the structure of formula I:

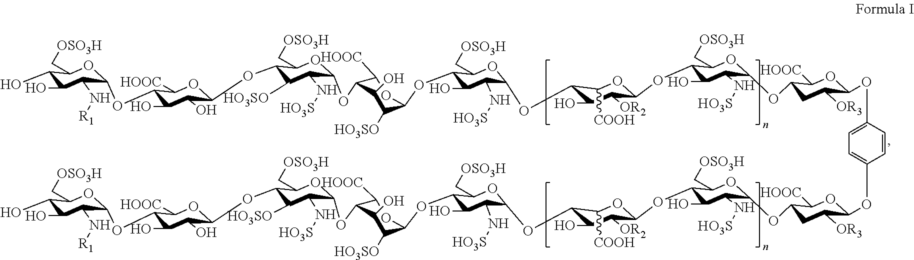

wherein R₁ is sulfonyl (—SO₃H) or acetyl (—COCH₃); R₂ and R₃ are sulfonyl or hydrogen (—H); and n is an integer from 0 to 5.

According to some embodiments of the present disclosure, n=0, 1 or 2.

According to some embodiments of the present disclosure, a phenyl linking heparin oligosaccharide may also be replaced with substituted phenyl, heteroaryl, substituted heteroaryl, C₁-C₅ alkyl or cycloalkyl.

According to some embodiments of the present disclosure, a substituent on the substituted phenyl and the substituted heteroaryl is selected from the group consisting of halogen, hydroxyl, nitro, trifluoromethyl, C₁-C₅ alkyl and cycloalkyl.

According to some embodiments of the present disclosure, the substituted phenyl is selected from the group consisting of:

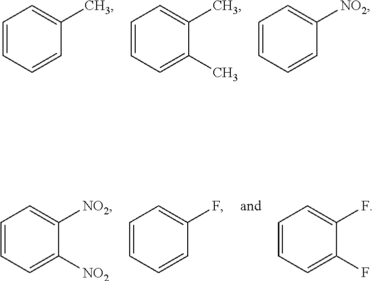

According to some embodiments of the present disclosure, the substituted heteroaryl is selected from the group consisting of:

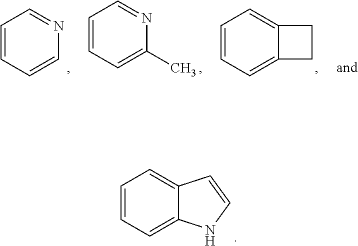

According to some embodiments of the present disclosure, the C₁-C₅ alkyl is

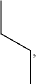

According to some embodiments of the present disclosure, the cycloalkyl is a cyclohexane.

According to some embodiments of the present disclosure, the anticoagulant phenyl-linked heparin oligosaccharide dimer is specifically one of the following compounds:

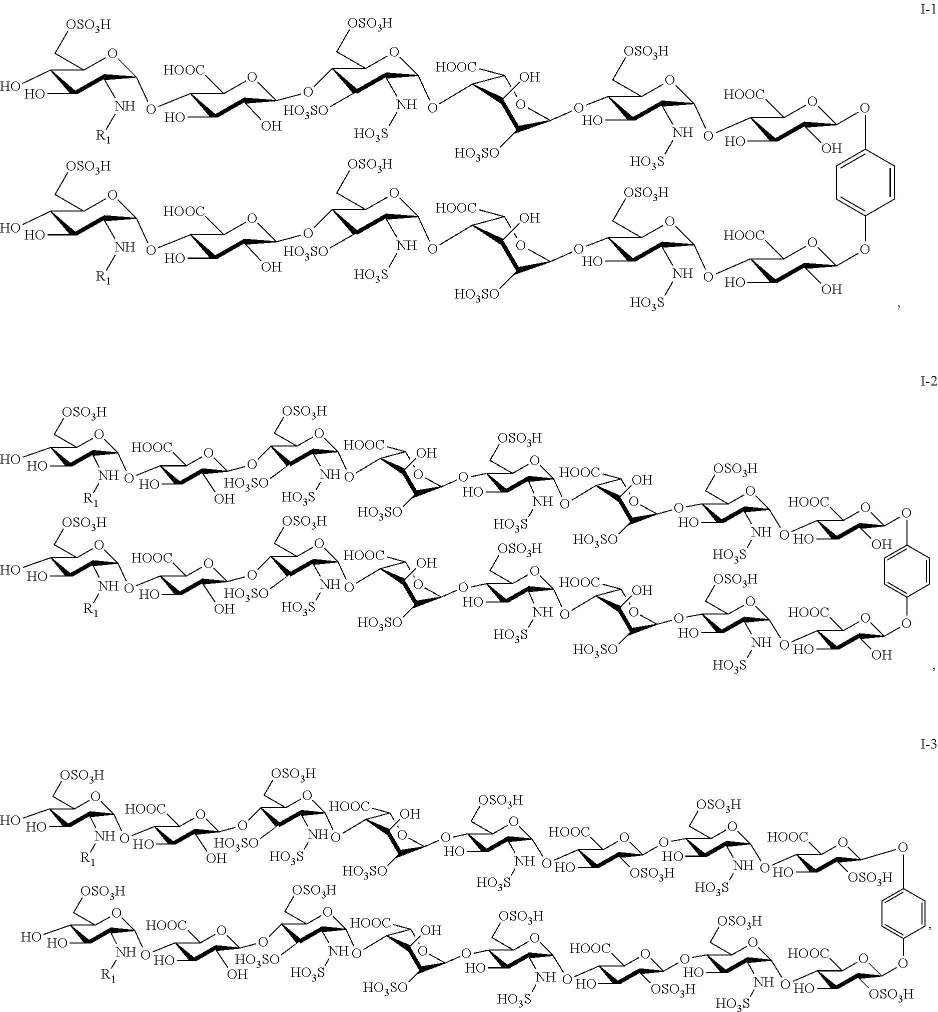

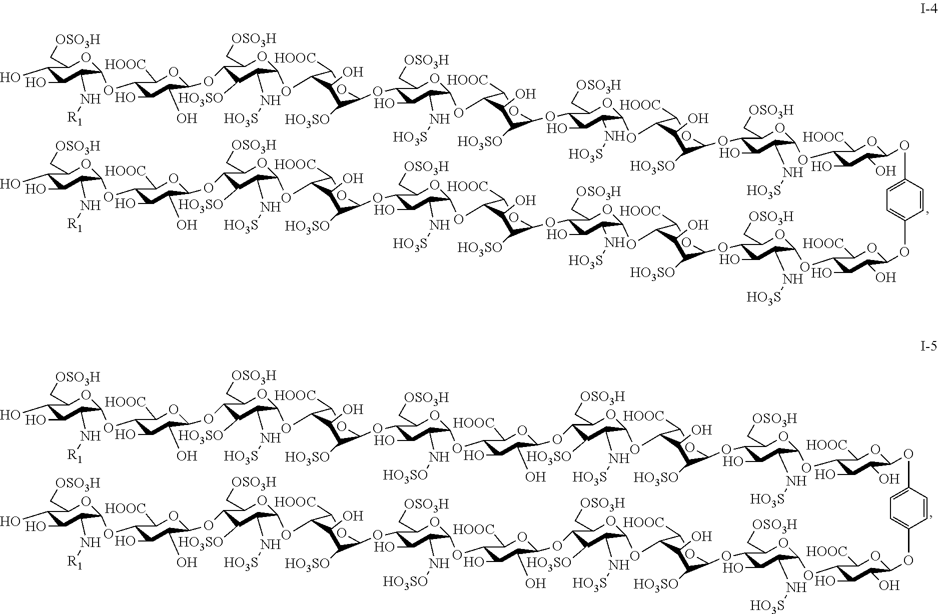

wherein, in formula I-1 to formula I-5, R₁ is sulfonyl (—SO₃H) or acetyl (—COCH₃).

According to some embodiments of the present disclosure, the anticoagulant phenyl-linked heparin oligosaccharide dimer molecule has significant anti-Factor Xa activity and no significant anti-Factor II a activity; it has similar pharmacokinetics profiles with short-chain heparin oligosaccharides, long half-life in vivo and high bioavailability; and it is not prone to cause adverse reaction of heparin-induced thrombocytopenia (HIT).

A second objective of the present disclosure is to provide a method for preparing the above anticoagulant phenyl-linked heparin oligosaccharide dimer, in which a chemoenzymatic synthesis strategy is adopted.

Provided is a method for preparing the above phenyl-linked heparin oligosaccharide dimer, where the method includes combining steps a and b that are directed to glycosyltransferase catalytic reactions and are repeated at least once with four or five steps of steps c, d, e, f and g that are directed to chemoenzymatic modification reactions, with a chemically synthesized dimer intermediate containing GlcA as a starting material;
step a, generating a saccharide chain-extended dimeric intermediate by transferring a GlcNTFA or GlcNAc residue from UDP-GlcNTFA or UDP-GlcNAc as a glycosyl donor to two non-reducing terminal GlcA residues of either a phenyl bis-O-glucuronide substrate or an oligosaccharide intermediate dimer having GlcA residues at non-reducing ends via α-1,4 glycosidic linkage catalyzed by N-acetylaminoglucosyltransferase (KfiA) or heparosan synthase 2 (PmHS2);
step b, generating a saccharide chain-extended intermediate by linking a GlcA residue from UDP-GlcA as a glycosyl donor to either glucosamine GlcNTFA or GlcNAc at non-reducing ends of a substrate via β-1,4 glycosidic linkage catalyzed by PmHS2;
step c, dissolving a heparin oligosaccharide dimeric intermediate in an alkaline aqueous solution, letting the alkaline aqueous solution to stand on ice to allow conversion of all the GlcNTFA residues in a saccharide chain into GlcNH₂ residues through cleavage of trifluoroacetylate (TFA), converting the GlcNH₂ residues to GlcNS residues under catalysis of N-sulfotransferase (NST) to obtain an N-sulfated heparin oligosaccharide dimeric intermediate;
step d, converting all GlcA residues in a dimeric saccharide chain located between two GlcNS residues into 2-O-sulfated iduronic acid (IdoA2S) under co-catalysis of C₅-epimerase (C₅-epi) and 2-O-sulfotransferase (2OST) to obtain an IdoA2S-containing heparin oligosaccharide dimeric intermediate;
step e, converting all specific GlcA residues in a substrate located between the two GlcNS residues into 2-O-sulfated glucuronic acid (GlcA2S) under catalysis of 2OST alone;
step f, generating a 6-O-sulfated heparin oligosaccharide dimeric intermediate by sulfating 6-OHs of all GlcNS or GlcNAc residues in a substrate saccharide chain under co-catalysis of 6-O-sulfotransferase 1 (6OST1) and 6-O-sulfotransferase 3 (6OST3); and
step g, subjecting 3-OH of GlcNS6S between GlcA and IdoA2S in the saccharide chain of the substrate to sulfation (GlcNS6S3S) under catalysis of 3-O-sulfotransferase 1 (3OST1) to obtain the anticoagulant phenyl-linked heparin oligosaccharide dimer as a target compound.

According to some embodiments of the present disclosure, a dimeric intermediate containing GlcA is phenyl bis-O-glucuronide or substituted phenyl bis-O-glucuronide, aromatic heterocyclic bis-O-glucuronide, substituted aromatic heterocyclic bis-O-glucuronide, C₁-C₅ alkyl bis-O-glucuronide or cycloalkyl bis-O-glucuronide.

According to some embodiments of the present disclosure, the phenyl bis-O-glucuronide as a starting material is prepared by the following process:
taking a fully acetylated glucose as a starting material, removing terminal acetyl using acetohydrazide to obtain an intermediate, reacting an exposed hydroxyl at anomeric position with trichloroacetonitrile (CNCCl₃) to obtain a glycosyl trichloroacetimidate ester, then subjecting the glycosyl trichloroacetimidate ester to glycosidation with para-diphenol under the participation of an accelerant to produce a intermediate, alcoholizing the intermediate to remove acetyls to obtain a phenyl bis-O-glucoside, and finally, selectively co-oxidating C₆—OH by PhI(OAc)₂ and Tempo to yield a target product.

The synthetic route is shown in Formula II below:

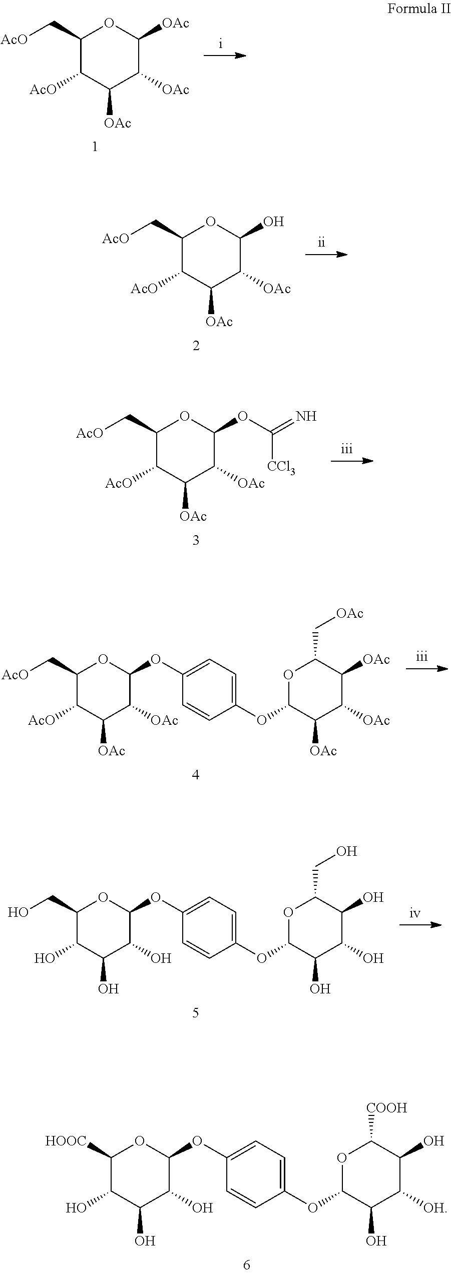

According to some embodiments of the present disclosure, a solvent for a reaction of removing terminal acetyl from the fully acetylated glucose 1 by acetohydrazide is anhydrous N,N-dimethylformamide (DMF) and the reaction is carried out at room temperature for 1 hour.

According to some embodiments of the present disclosure, the exposed hydroxyl at anomeric position of the intermediate 2 reacts with trichloroacetonitrile (CNCCl₃) at less than 0° C. for more than 3 hours, and a solvent for the reaction is a mixed solution of 1,8-diazabicyclo[5.4.0]undec-7-ene (DBU) and anhydrous dichloromethane (DCM). The DBU and anhydrous DCM may be mixed in any ratio.

According to some embodiments of the present disclosure, a reaction of glycosyl trichloroacetimidate 3 with para-diphenol is performed at −5° C. for 1 hour using anhydrous DCM containing molecular sieve as a solvent and BF₃-Et₂O as an accelerant, a resulting reaction solution is extracted by ethyl acetate (EA), dried in vacuum and recrystallized from methanol to obtain an intermediate 4; the intermediate 4 is treated with anhydrous MeOH-sodium methoxide at room temperature for more than 1 hour to remove the acetyls, neutralized with acidic resin to neutrality, filtered, dried in vacuum and crystallized to obtain an intermediate 5. Each reagent is used in an amount according to the prior art to which the present disclosure pertains.

According to some embodiments of the present disclosure, C₆—OH of the intermediate 5 is selectively co-oxidized by both PhI(OAc)₂ and Tempo, and a solvent used is a mixed solution of t-BuOH, DCM and H₂O in a volume ratio of 4:4:1, the reaction is held overnight at room temperature and recrystallized from acetonitrile to obtain a target product phenyl bis-O-glucuronide 6.

Each reagent is used in an amount according to the prior art to which the present disclosure pertains.

According to some embodiments of the present disclosure, the anticoagulant phenyl-linked heparin oligosaccharide dimer is prepared by the following method: a target compound I-1 is prepared by adopting the method of the step a, the step b, the step c, the step d, the step f and the step g using phenyl bis-O-glucuronide 6 as a raw material, and a synthetic route is shown in Formula III:

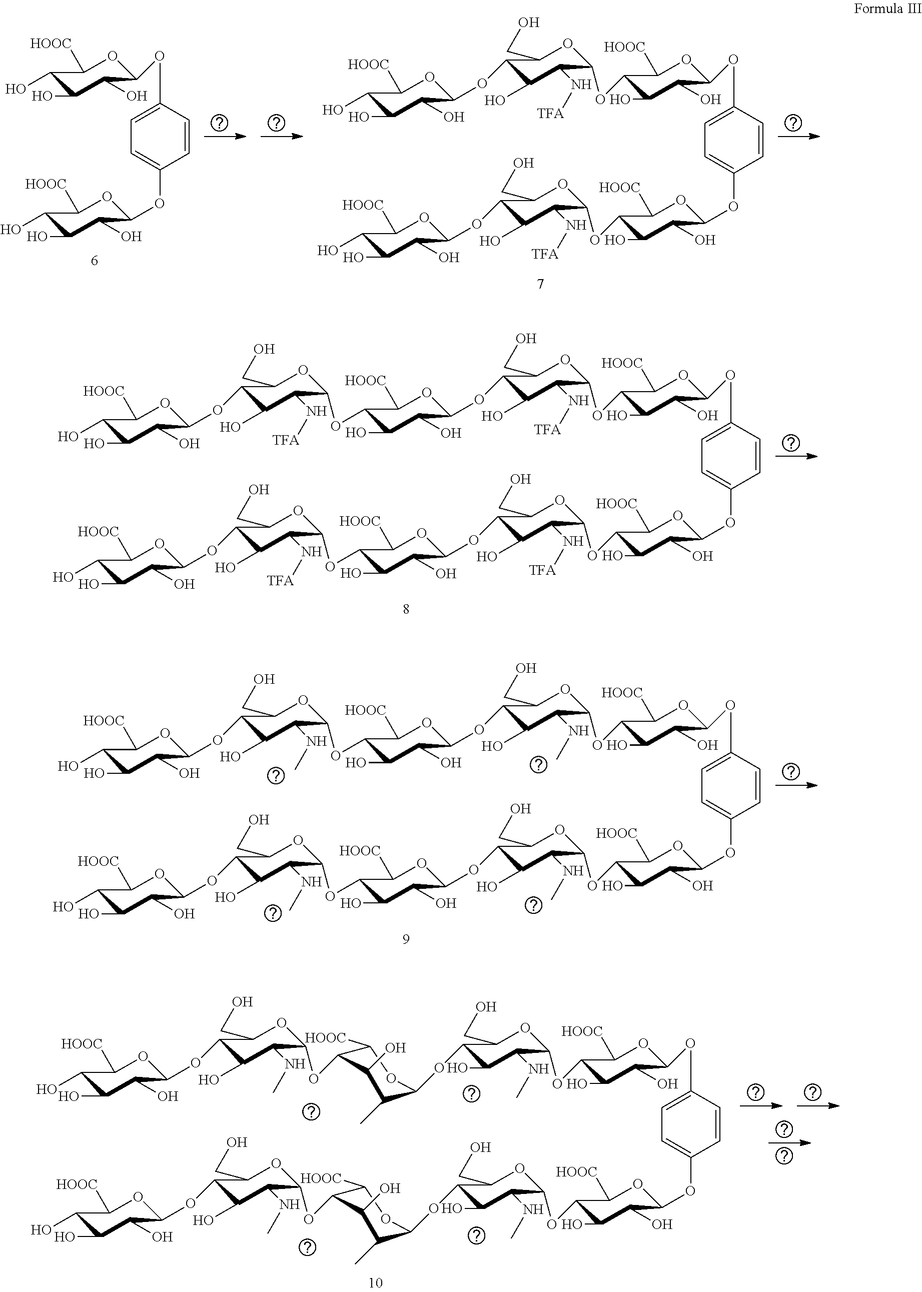

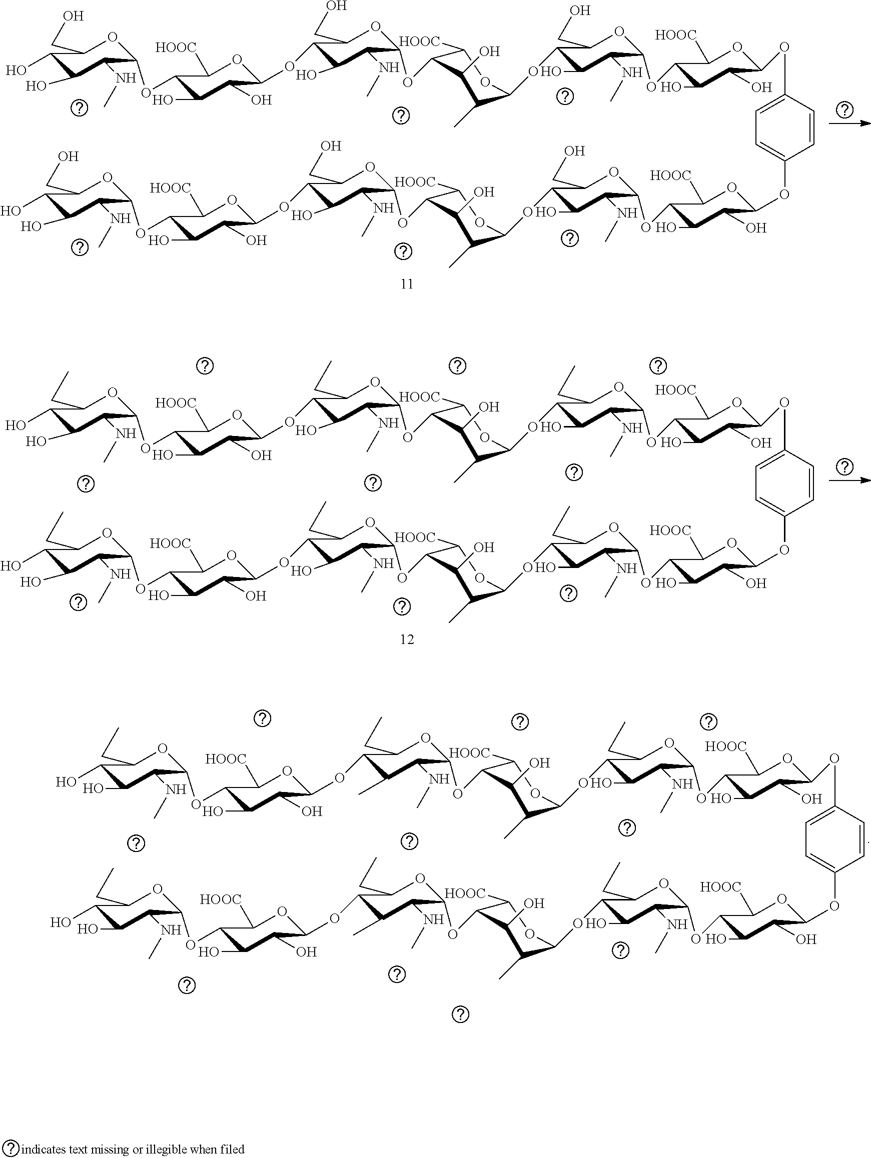

The chemoenzymatic synthetic route of the formula I-1 may be recorded briefly as: a→b→a→b→c→d→a→optional c→f→g.

According to some embodiments of the present disclosure, synthetic routes for the compounds of formula I-2 to formula I-5 are as follows:
I-2: a→b→a→b→c→d→a→b→c→d→a→optional c→f→g;
I-3: a→b→a→b→c→e→a→b→c→d→a→optional c→f→g;
I-4: a→b→a→b→c→d→a→b→c→d→a→b→c→d→a→optional c→f→g; and
I-5: a→b→a→b→c→d→f→a→b→a→b→c→d→a→optional c→f g;

According to some embodiments of the present disclosure, the KfiA and the PmHS2 are derived from Escherichia coli K5 and Pasteurella multocida, respectively, expressed by Escherichia coli, and a saccharide chain extension reaction catalyzed by enzyme is performed at 20° C. to 37° C. using a buffer including 50 mmol/L Tris-HCl with a pH value of 7.0 to 7.5, and a resulting enzymatic product is purified with a reversed-phase chromatography with C18 column or an anion exchange chromatography to yield a purified product.

The amount of the enzymes, the substrates added and the reaction time are determined according to the prior art to which the present disclosure pertains.

According to some embodiments of the present disclosure, the heparin-modifying enzymes NST, C₅-epi, 2OST, 6OST1, 6OST3 and 3OST1 are obtained by recombinant expression in Escherichia coli, yeast or insect cells, and catalytic reactions by the heparin-modifying enzymes NST, 2OST, 6OST1, 6OST3 and 3OST1 is conducted at 20° C. to 37° C. using 50 mmol/L 2-(N-morpholino) ethanesulfonic acid (MES) with a pH value of 7.0 to 7.5 as a buffer, and 3′-phosphoadenosine-5′-phosphosulfate (PAPS) as a sulfate group donor, and a resulting enzymatic solution is purified with an anion exchange chromatography to yield a product.

The amount of the enzymes and substrates added and the reaction time are determined according to the prior art to which the present disclosure pertains.

A third objective of the present disclosure is to provide use of the anticoagulant phenyl-linked heparin oligosaccharide dimer containing AT binding sequence in the preparation of anticoagulant and antithrombotic drugs with powerful efficacy, wherein the anticoagulant phenyl-linked heparin oligosaccharide dimer containing AT binding sequence has specific anti-Factor Xa activity, no significant anti-Factor II a activity, neutralizable anticoagulant activity by protamine sulfate, and excellent pharmacokinetics

Provided is an anticoagulant and antithrombotic pharmaceutical composition including the above phenyl-linked heparin oligosaccharide dimer containing AT binding sequence and one or more of a pharmaceutically acceptable carrier or an excipient.

The ratio of the phenyl-linked heparin oligosaccharide dimer to the pharmaceutically carrier or the excipient is arbitrary.

In the present disclosure, an anticoagulant phenyl-linked heparin oligosaccharide dimer containing AT binding sequence is successfully synthesized by a chemoenzymatic method with two glucuronic acid molecules covalently linked to a same benzene ring as starting materials for the first time. It not only exhibits powerful anticoagulant activity, but also demonstrates characteristics similar to long-chain heparin molecules in terms of protamine neutralization capability, while possessing desirable pharmacokinetic profiles similar to short-chain heparin molecules. Furthermore, it has a low risk of inducing adverse reactions such as HIT is low, requires a minimal number of steps, as few as 9 steps, and may be developed into a new generation of safer and higher quality anticoagulant innovative drug with independent intellectual property, holding an important application value.

Technical features and advantages of the embodiments of the present disclosure:
1. It is confirmed in the present disclosure for the first time that various heparin oligosaccharide (hexasaccharide, octasaccharide and decasaccharide) dimer molecules containing AT binding sequence are successfully prepared via 9-18 steps of chemoenzymatic reaction with phenyl bis-O-glucuronide containing two GlcAs as starting materials.
2. The novel pheneyl-linked heparin oligosaccharide dimer containing AT-binding sequence of the present disclosure shows significant anti-Factor Xa activity and no significant anti-Factor II a activity as determined by the chromogenic substrate assay. It is demonstrated for the first time that the two AT-binding sequences in the phenyl-linked heparin oligosaccharide dimer are able to activate the AT without interfering with each other, and that its half maximal inhibitory molar concentration (IC₅₀) against Factor Xa is significantly lower than that of positive controls including Fondaparinux and long chain heparin dodecasaccharide molecules reported by Jian Liu.
3. The specific anti-Factor Xa activity of the novel phenyl-linked heparin oligosaccharide dimer containing AT-binding sequence of the present disclosure may be efficiently neutralized by protamine sulfate with a neutralization rate of >70%, which is comparable to that of long-chain ungraded heparin UFH and reported heparin dodecasaccharides, whereas the anti-Factor Xa activity of commercially available Fondaparinux can hardly be neutralized by protamine sulfate, and none of the reported existing anticoagulant heparin hexasaccharides, octasaccharides and decasaccharides can be neutralized by protamine sulfate. Therefore, the phenyl-linked heparin oligosaccharide dimer of the present disclosure has similar properties to long chain heparin molecules of heparin dodecasaccharide and longer, and is capable of forming higher affinity with protamine sulfate so as to be neutralized by protamine sulfate.
4. The phenyl-linked heparin oligosaccharide dimer of the present disclosure has desirable pharmacokinetic profiles, e.g., the in vivo half-life of Compound I-1-1 is comparable to that of commercially available Fondaparinux, and is significantly superior to that of animal-derived heparin and reported existing heparin decasaccharide molecules.
5. The portions of the saccharide chain and modification groups outside the AT-binding sequence in the structure of the phenyl-linked heparin oligosaccharide dimer in the present disclosure has less influence on the anti-Factor Xa activity, but has more pronounced effects on the efficiency of protamine neutralization and pharmacokinetic profiles.
6. The phenyl-linked heparin oligosaccharide dimer in the present disclosure may be used for the preparation of potent anticoagulant and antithrombotic drugs that are cost-effective, of higher quality, and safer.

### DESCRIPTION OF THE DRAWINGS

FIG. 1 shows the ¹H NMR spectrum of the phenyl bis-O-glucuronide 6 prepared in Example 1;

FIG. 2 shows a high performance liquid chromatography (A) and a mass spectrometry (B) of the phenyl-linked heparin hexasaccharide dimer I-1-1 prepared in Example 5;

FIG. 3 shows the ¹H NMR spectrum (A) and HSQC spectrum (B) of the phenyl-linked heparin hexasaccharide dimer I-1-1 prepared in Example 5;

FIG. 4 is a diagram showing the in vitro anti-factor Xa of the phenyl-linked heparin hexasaccharide dimer I-1-1 prepared in Example 5;

FIG. 5 is a diagram showing the in vitro neutralization effect of protamine on the anticoagulant activity of the phenyl-linked heparin hexasaccharide dimer I-1-1 of Example 5; and

FIG. 6 is a diagram showing the in vivo pharmacokinetic profiles of the phenyl-linked heparin hexasaccharide dimer I-1-1 prepared in Example 5.

### SPECIFIC IMPLEMENTATIONS

The present disclosure will be further described with specific examples; however, the protection scope of the present disclosure is not limited thereto. The numbers of the target compounds in the following examples are the same as those in Table 1. Unless otherwise specified, the drugs and reagents involved in the examples are all common commercial products.

### Example 1: Chemical Synthesis of the Phenyl Bis-O-Glucuronide 6

Compound 1 (1 g, 2.6 mmol) and AcOH/NH₂—NH₂ (1.4 equiv., 3.6 mmol, 330 mg) were dissolved in 5 mL of anhydrous DMF and reacted at room temperature for 1 hour. After the reaction, the resulting reaction solution was diluted with EA, the organic layer was washed with H₂O and saturated salt solution respectively, dried in vacuum to obtain the filter residue. After purification by silica gel column chromatography (petroleum ether (PE):EA=2:1), 752 mg of colorless oily liquid 2 was obtained, with a yield of 84.3%.

Compound 2 (752 mg, 2.16 mmol) was dissolved in 5 ml of anhydrous DCM, then CNCCl₃ (3 equiv., 6.5 mmol, 0.65 ml) and DBU (0.2 equiv., 0.43 mmol) were added in sequence at low temperature, and the reaction was carried out at this temperature for 3 hours. After the reaction, the resulting reaction solution was directly dried in vacuum to obtain filter residue, and 893 mg of colorless oily liquid 3 was obtained by silica gel column chromatography (PE:EA=4:1), with a yield of 83.8%.

Compound 3 (893 mg, 1.8 mmol, 2.2 equiv.) and para-diphenol (0.82 mmol, 83 mg) were dissolved in 10 mL of anhydrous DCM containing a proper amount of molecular sieve, BF₃-Et₂O (4 equiv., 3.28 mmol, 0.4 ml) was added at −5° C., and the reaction continued at this temperature for 1 hour. After the reaction, the resulting reaction solution was diluted with EA, and the organic layer was washed with saturated sodium bicarbonate solution, water and saturated salt solution in turn. The washed organic layer was dried in vacuum, and the obtained residue was recrystallized from methanol to obtain 462 mg of white solid compound 4 with a yield of 73%. ¹HNMR (400 MHz, Chloroform-d) δ 6.91 (d, J=4.6 Hz, 4H), 5.30-5.11 (m, 6H), 4.98 (dd, J=7.4, 3.6 Hz, 2H), 4.26 (dd, J=12.4, 5.2 Hz, 2H), 4.15 (dt, J=12.2, 2.8 Hz, 2H), 3.80 (ddt, J=10.0, 5.2, 2.4 Hz, 2H), 2.04 (dt, J=13.2, 4.8 Hz, 24H).

Compound 4 (462 mg, 0.6 mmol) and sodium methoxide (0.1 equiv. 3 mg) were dissolved in 10 mL of anhydrous MeOH and reacted at room temperature for 1 hour. After the reaction, acidic resin was added and stirred for 10 min, and the resulting reaction solution was neutralized to neutrality, filtered, and dried in vacuum to remove most of the solvent. The remaining reaction solution was put into the refrigerator for precipitation to obtain 247 mg of white solid compound 5, with a yield of 95%.

Compound 5 (247 mg, 0.57 mmol), PhI(OAc)₂ (8 equiv., 4.6 mmol, 1.5 g) and Tempo (4 equiv. 2.3 mmol, 355 mg) were dissolved in 18 mL mixed solvent (t-BuOH:DCM:H₂O=4:4:1), reacted overnight. After the reaction, the resulting reaction solution was diluted by EA, the organic layer was washed with water, the water layer was spin-dried, and the vacuum dried residue was recrystallized from acetonitrile to obtain 162 mg of white solid compound 6 with a yield of 62%.

The synthetic route is shown in the following formula II:

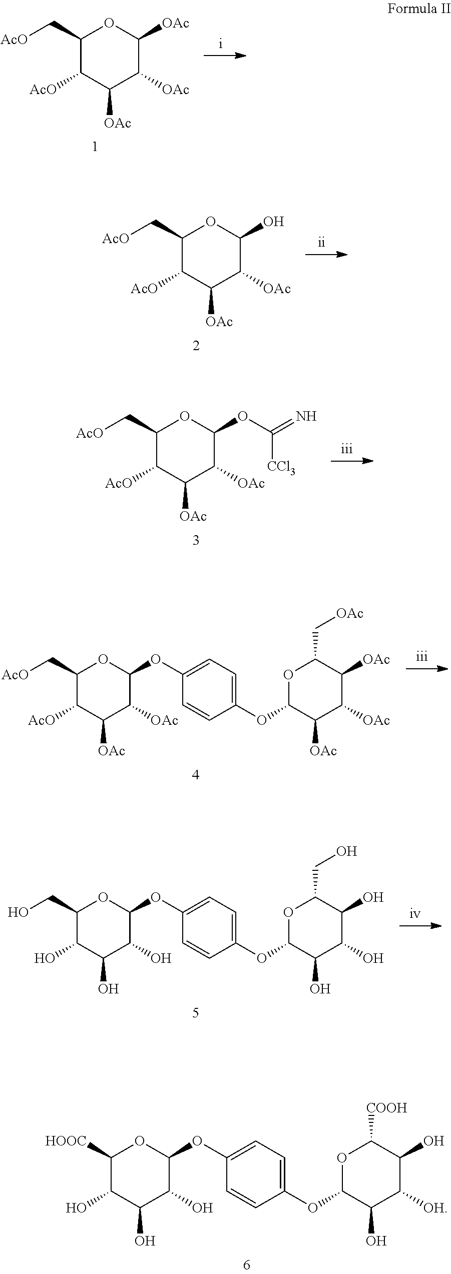

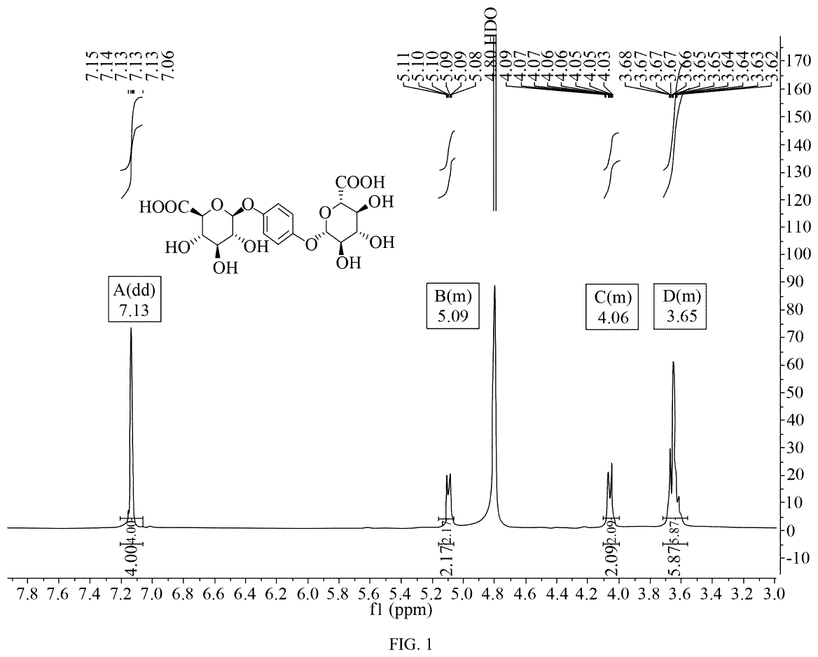

The ¹H NMR spectrum of compound 6 is shown in FIG. 1.

The important data are shown. ¹H NMR (400 MHz, D₂O) δ 7.13 (dd, J=2.8, 1.8 Hz, 4H), 5.16-5.06 (m, 2H), 4.10-4.00 (m, 2H), 3.72-3.56 (m, 6H).

### Example 2: Enzymatic Synthesis of Heparin Pentasaccharide Backbone Dimer 8

Fifty milligrams of phenyl bis-O-glucuronide 6 was weighed and dissolved in about 50 mL of 50 mmol/L Tris-HCl buffer (containing 6 mmol/L MnCl₂, pH=7.2), UDP-GlcNTFA in an amount of 2.2 times equivalent to the substrate, into which 2.5 mL of KfiA enzyme were added, and the solution was stirred overnight at room temperature. The reaction solution was detected by polyamine-based anion exchange-high-performance liquid chromatography (PAMN-HPLC), and the chromatographic conditions included: 0→100% KH₂PO₄ gradient elution within 45 min at a flow rate of 0.5 m/min, with a detection wavelength of 280 nm. The pH of the reaction was adjusted to 2-3 with TFA until the reaction yield ≥99% to stop the reaction, purified with C₁₈ column (3×50 cm), and eluted with methanol-water containing 0.1% TFA to collect the target fraction. The obtained product was placed into 50 mL of the same buffer as above, into which 2.2 times equivalent of UDP-GlcA and 3 mL of PmHS2 enzyme were added, and the mixture was stirred overnight at room temperature. The reaction was detected by PAMN-HPLC and purified with Cis column until the yield was ≥99% to obtain the intermediate 7. The above enzymatic reactions catalyzed by KfiA and PmHS2 were repeated to obtain the heparin pentasaccharide backbone dimer 8, whose molecular weight was consistent with the theory as measured by ESI-MS.

The synthetic route is shown in the following formula IV:

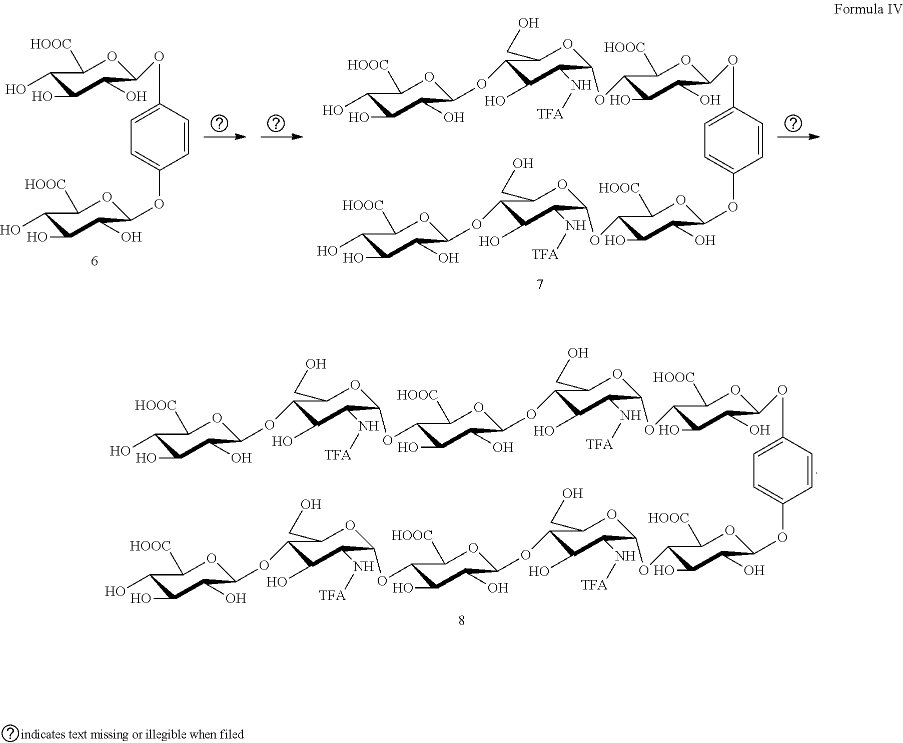

### Example 4: Chemoenzymatic Synthesis of Phenyl-Linked Heparin Pentasaccharide Dimer 10 Containing IdoA2S

The pentasaccharide backbone dimer 8 was dissolved in 100 mL deionized water, placed on ice, and 0.5 mol/L LiOH solution was added dropwise to pH=12. The mixture was kept in ice bath for 2 hours, and the reaction progress was monitored by PAMN-HPLC; after the reaction, the pH was adjusted to neutrality with glacial acetic acid, a proper amount of 1 mol/L 2-(N-morpholino) ethanesulfonic acid (MES) solution (pH=7.4) was added to make a final concentration of 50 mmol/L, while 4.4 times equivalent of PAPS and 3 mL NST were added, and stirred overnight at room temperature. The reaction solution was detected by PAMN-HPLC. The pH of the reaction was adjusted to 4-5 with acetic acid when the reaction yield is more than 95% to stop the reaction, followed by purification by Q-Sepharose column (1×20 cm) at a flow rate of 3 mL/min, and elution with 0→100% buffer containing 1 mol/L NaCl and 50 mmol/l NaAc (pH=5). The detection wavelengths were 260 nm and 280 nm, and the target fraction was collected, desalinated and dried to obtain the N-sulfated product 9. The molecular weight was 2130.22 Da by ESI-MS.

The above product 9 was added into a 50 mol/L MES buffer with pH=7.0 to 7.4, into which 2 mmol/L CaCl₂ and an appropriate amount of C₅-epi enzyme were added, and the reaction volume was adjusted to 100 mL. The reaction was conducted in water bath at 37° C. for 2 hours. Then, about 2.5 times equivalent of PAPS, extra C₅-epi and sufficient amount of 2OST enzyme were added, and the reaction was carried out overnight at room temperature. The reaction solution was detected by PAMN-HPLC, and enzyme or PAPS was added as needed until the reaction was over. The resulting reaction solution was purified by Q-Sepharose strong anion column (1×20 cm) to obtain the heparin pentasaccharide dimer 10. Its molecular weight was 2290.20 Da as measured by ESI-MS.

The synthetic route is shown in the following formula V:

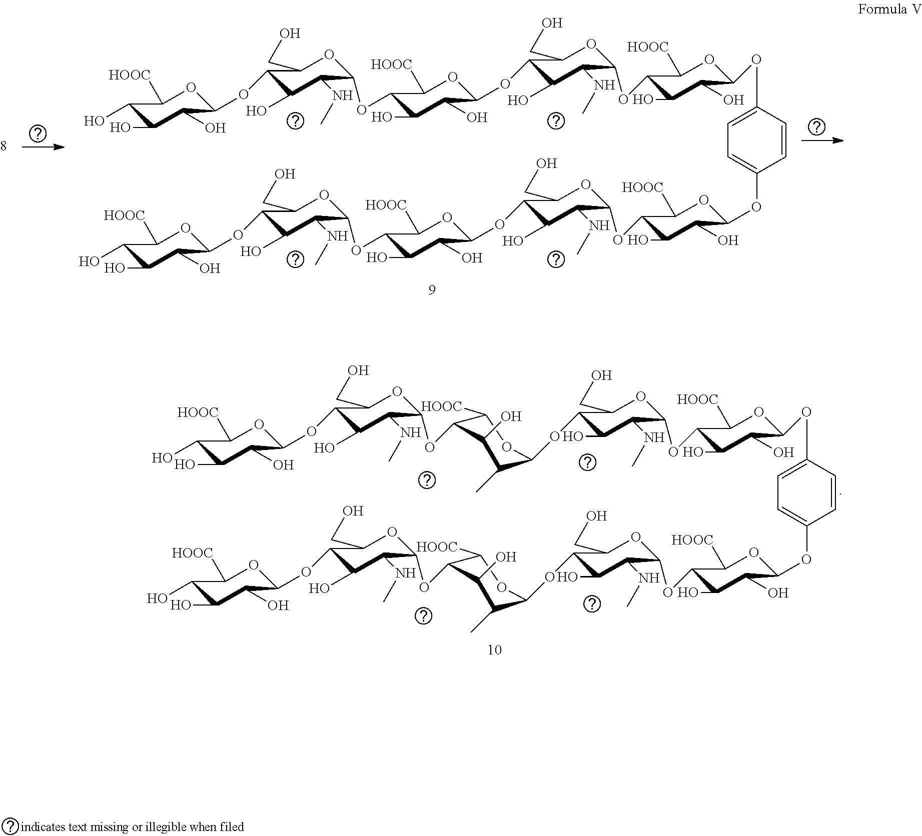

### Example 5: Preparation of Phenyl-Linked Heparin Hexasaccharide Dimer I-1-1 by Chemoenzymatic Method

The pentasaccharide dimer 10 was used as substrate, the saccharide chain was extended by A enzyme as described in Example 1, and the resulting reaction solution was purified by Q-Sepharose strong anion column (1 cm×20 cm). The obtained product was modified by a chemoenzymatic method of N-sulfation as described in Example 4 to obtain the phenyl-linked heparin hexasaccharide dimer 11. The molecular weight was 2772.41 Da as measured by ESI-MS, which was consistent with the theoretical value.

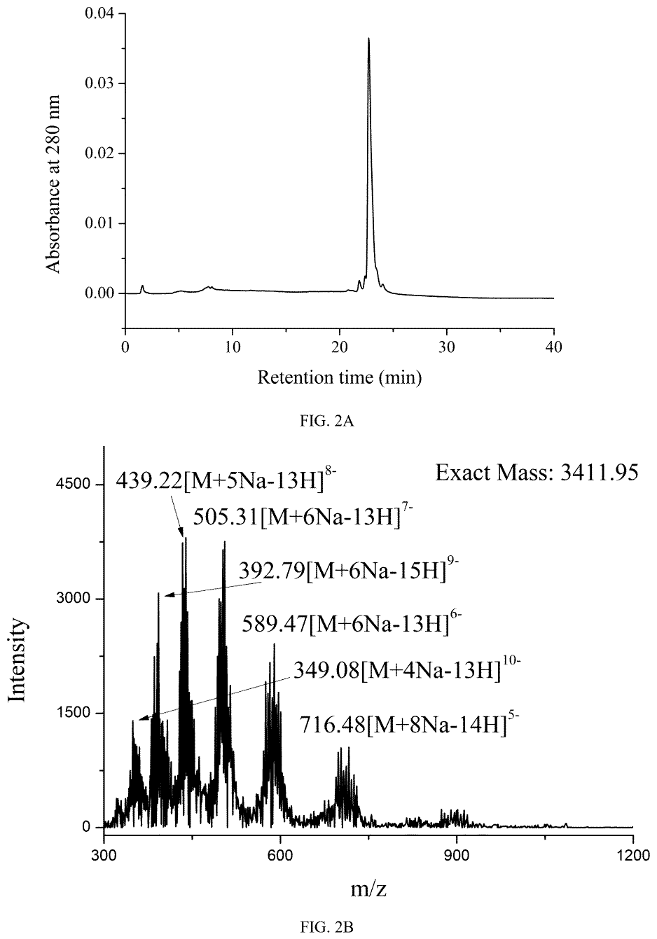

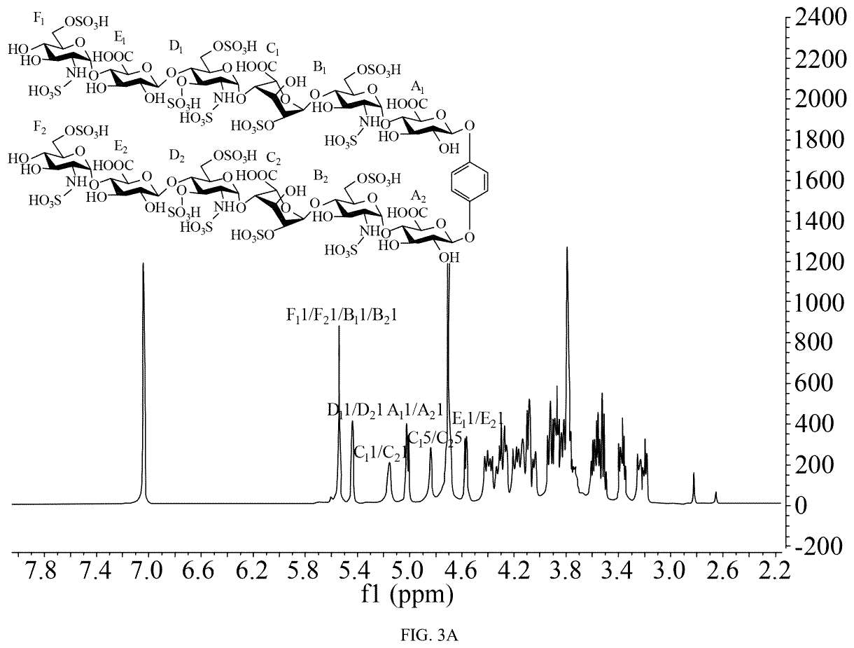

The phenyl-linked hexasaccharide dimer 11 was placed in 50 mmol/L MES buffer with pH=7.0 to 7.5, and 7 times equivalent of PAPS, 4 mL of 6OST1 and 4 mL of 6OST3 were added; the reaction volume was adjusted to 140 mL, and the reaction was carried out in water bath at 37° C. overnight. The reaction progress was monitored by strong anion exchange high-performance liquid chromatography (SAX-HPLC) and enzyme or PAPS was added as needed. Chromatographic conditions were as follows: the flow rate was 1 m/min, gradient elution was carried out with 0→100AP eluent B (50 mmol L NaAc+2 mol/L NaCl, pH=5), and the detection wavelengths were 260 nm and 280 nm. The pH of the reaction was adjusted to 4-5 with dilute acetic acid when the reaction yield was >99% to terminate the reaction, and the enzyme was removed by freezing and thawing at a −20° C. refrigerator without purification. The pH of the solution was adjusted to 7.0 to 7.5, followed by addition of about 2.5 times equivalent of PAPS and 5 mL of 3OST1 enzyme to adjust the reaction volume to 200 mL, and the reaction was conducted overnight in water bath at 37° C. The reaction solution was monitored by PAMN-HPLC until the substrate modification rate was above 99%. The pH of the reaction solution was adjusted to 4-5 with dilute acetic acid, and then the target phenyl-linked heparin hexasaccharide dimer I-1-1 was purified by Q-Sepharose strong anion column (1 cm×10 cm). The purity was up to 92% (FIG. 2A). The molecular weight was 3411.99 Da as measured by ESI-MS (FIG. 2B), which was consistent with the theoretical value. The structure thereof was characterized by ¹H NMR and HSQC (FIG. 3).

The synthetic route is shown in the following formula VI:

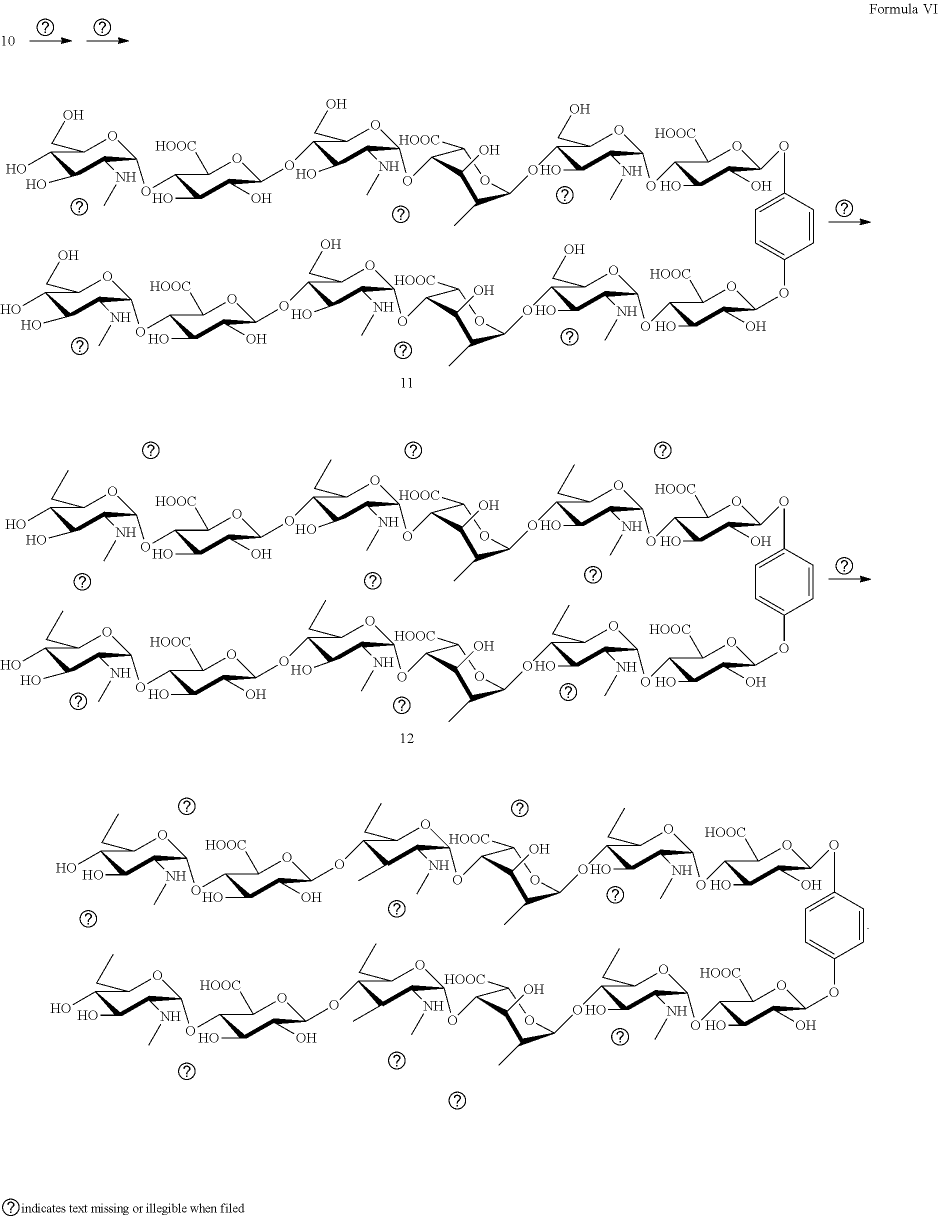

### Test Example 1: Determination of In Vitro Anticoagulant Activity of Phenyl-Linked Heparin Hexasaccharide Dimer I-1-1

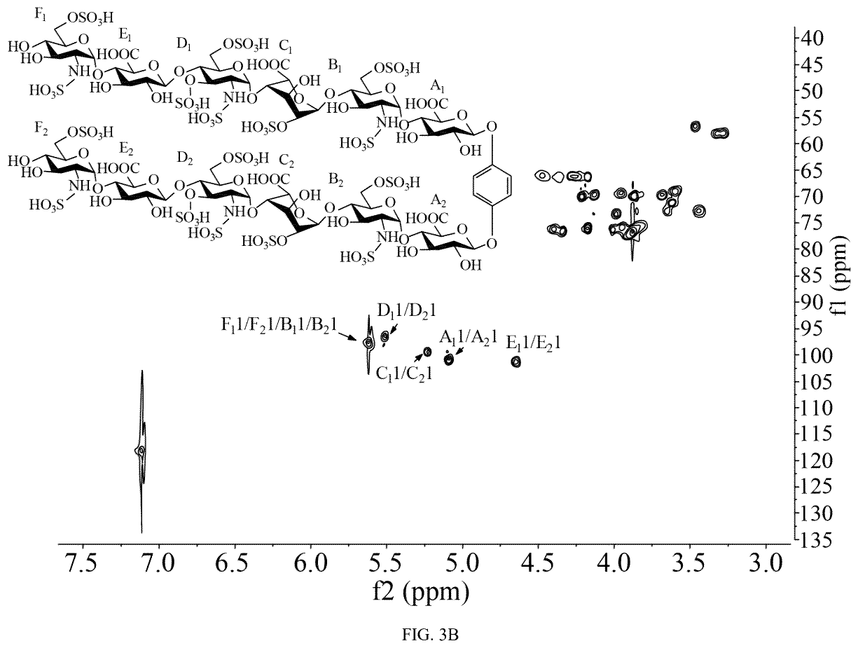

The IC₅₀ value of the anti-Factor Xa activity of the phenyl-linked heparin hexasaccharide dimer I-1-1 prepared in Example 5 as determined by the chromogenic substrate assay was 7.2 nmol/L (28.15 ng/mL), and the IC₅₀ values of unfractionated heparin (UFH) and Fondaparinux under the same conditions were 378.3 ng/mL and 10.8 nmol/L (18.63 ng/mL), respectively. In other words, the IC₅₀ value of the anti-Factor Xa activity of the compound I-1-1 by molar concentration was significantly lower than that of Fondaparinux, indicating that it was a potent Xa inhibitor, as shown in FIG. 4. At the same time, the target compound I-1-1 was similar to Fondaparinux as detected by the chromogenic substrate method, in terms of having no significant anti-Factor Ha activity (data not shown). Therefore, the phenyl-linked heparin hexasaccharide dimer I-1-1 prepared by the present disclosure is a specific inhibitor of Factor Xa. Moreover, because the two AT binding sequences contained in the dimer can activate AT without interference with each other, the dimer is superior to Fondaparinux and heparin dodecasaccharides reported by Jian Liu with a single AT binding sequence, and its half maximal inhibitory molar concentration (IC₅₀) is significantly lower than that of the positive control Fondaparinux and long-chain heparin dodecasaccharides reported by Jian Liu.

### Test Example 2: Determination of Neutralization of Anticoagulant Activity of the Phenyl-Linked Heparin Hexasaccharide Dimer I-1-1 by Protamine Sulfate

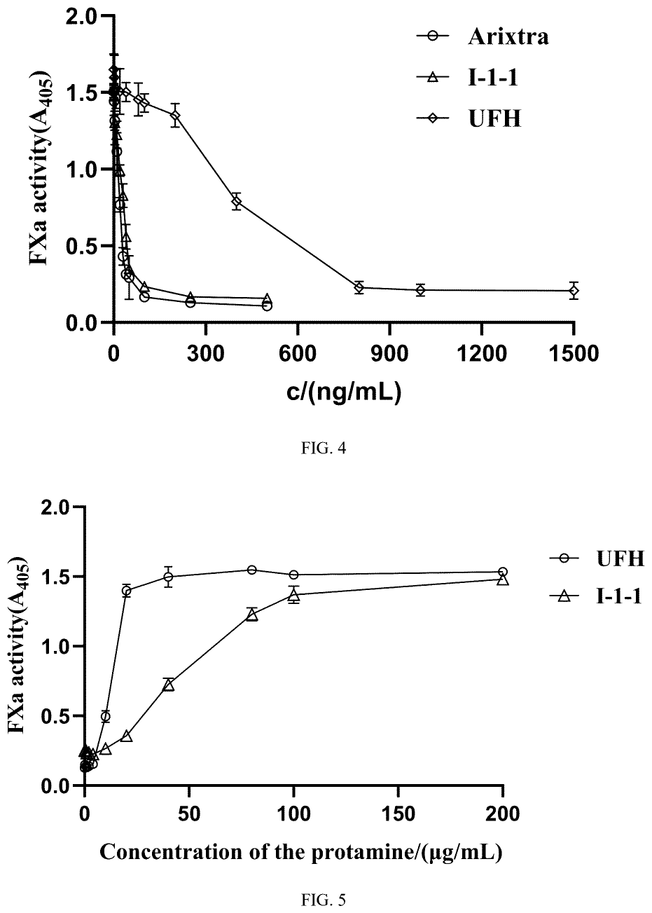

The effects of different concentrations of protamine sulfate on the anti-Factor Xa activity of the phenyl-linked heparin hexasaccharide dimer I-1-1 prepared in Example 5 were determined by the chromogenic substrate method. The results are shown in FIG. 5. It can be seen from FIG. 5 that the in vitro anti-Factor Xa activity of the phenyl-linked heparin hexasaccharide dimer I-1-1 was almost completely reversed by protamine sulfate, a phenomenon similar to that of UFH. Therefore, the phenyl-linked heparin hexasaccharide dimer I-1-1 prepared by the present disclosure is a novel heparin analog whose anticoagulant activity may be neutralized by protamine sulfate.

### Test Example 3: Pharmacokinetic Profiles of the Phenyl-Linked Heparin Hexasaccharide Dimer I-1-1 In Vivo

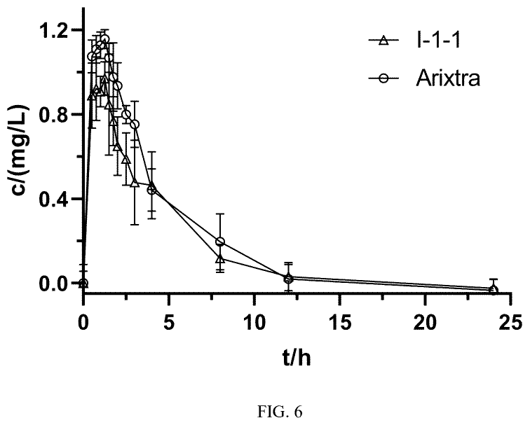

Taking low molecular weight heparin standard as an example, a standard curve was established by the chromogenic substrate method, and the titer of Fondaparinux and the phenyl-linked heparin hexasaccharide dimer I-1-1 was 1,290.9 I/mg and 1,121.5 IU/mg, respectively. Both Fondaparinux and the phenyl-linked heparin hexasaccharide dimer I-1-1 were injected subcutaneously into Wister male rats weighing 200±20 g (n≥3) at a dose of 300 IU/kg. Blood samples were collected from the jugular vein of rats at 0, 0.5, 0.75, 1, 1.25, 1.5, 1.75, 2, 2.5, 3, 4, 8, 12 and 24 hours respectively (based on the actual blood collection time). After the blood samples were centrifuged at 5000 r/min for 15 minutes, the supernatant was taken to determine the anti-Factor Xa activity by the above method, and the residual concentrations of Fondaparinux and the phenyl-linked heparin hexasaccharide dimer I-1-1 in rats were calculated. The drug concentration vs. time curve was drawn. As shown in FIG. 6, it can be seen that the curve of Fondaparinux is very similar to that of the phenyl-linked heparin hexasaccharide dimer I-1-1 of the present disclosure. The data was further processed with DAS 2.0 software, and it can be obtained that the t_{1/2} of the heparin hexasaccharide benzene dimer I-1-1 is up to 2.269 h, which is slightly longer than that of Fondaparinux with the t_{1/2} of 2.166 h, indicating that the half-life of the phenyl-linked heparin hexasaccharide dimer I-1-1 is excellent.

What is claimed is:

1. An anticoagulant phenyl-linked heparin oligosaccharide dimer or a pharmaceutically acceptable salt of the anticoagulant phenyl-linked heparin oligosaccharide dimer, having a structure of formula I: 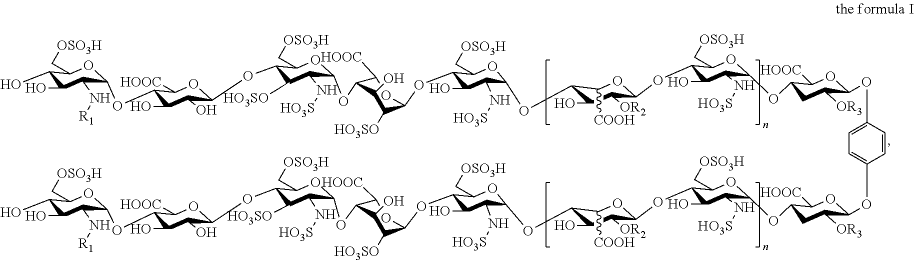
wherein
R₁ is sulfonyl (—SO₃H) or acetyl (—COCH₃);
R₂ and R₃ are the sulfonyl or hydrogen (—H); and
n is an integer from 0 to 5.

2. The anticoagulant phenyl-linked heparin oligosaccharide dimer or the pharmaceutically acceptable salt of the anticoagulant phenyl-linked heparin oligosaccharide dimer according to claim 1, wherein
n=0, 1, or 2,
a phenyl linking heparin oligosaccharide is optionally replaced with substituted phenyl, heteroaryl, substituted heteroaryl, C₁-C₅ alkyl, or cyclohexyl, and
a substituent on the substituted phenyl and the substituted heteroaryl is selected from the group consisting of halogen, hydroxyl, nitro, trifluoromethyl, the C₁-C₅ alkyl, and the cyclohexyl.

3. The anticoagulant phenyl-linked heparin oligosaccharide dimer or the pharmaceutically acceptable salt of the anticoagulant phenyl-linked heparin oligosaccharide dimer according to claim 2, wherein the substituted phenyl is selected from the group consisting of: 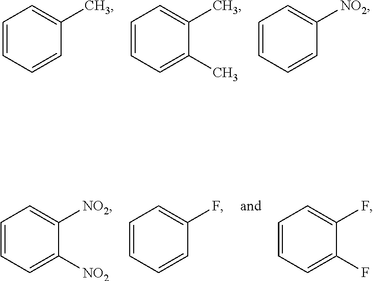
the substituted heteroaryl is selected from the group consisting of: 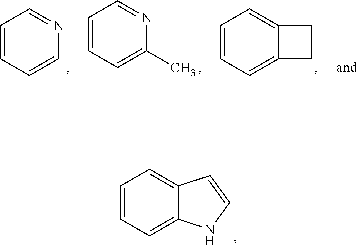
the C₁-C₅ alkyl is 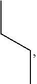
and
the cyclohexyl is cyclohexane.

4. The anticoagulant phenyl-linked heparin oligosaccharide dimer or the pharmaceutically acceptable salt of the anticoagulant phenyl-linked heparin oligosaccharide dimer according to claim 1, wherein
the anticoagulant phenyl-linked heparin oligosaccharide dimer is one of the following compounds: 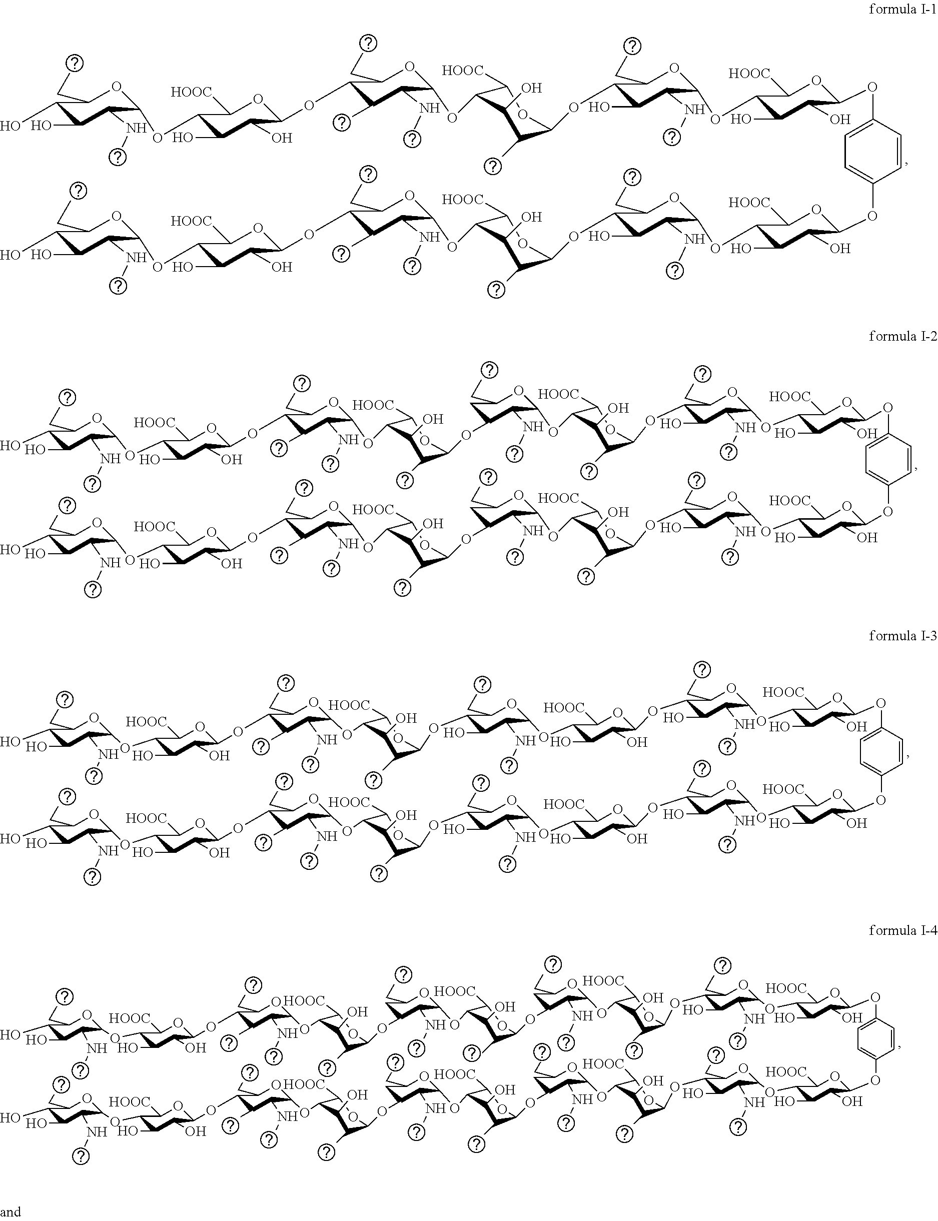 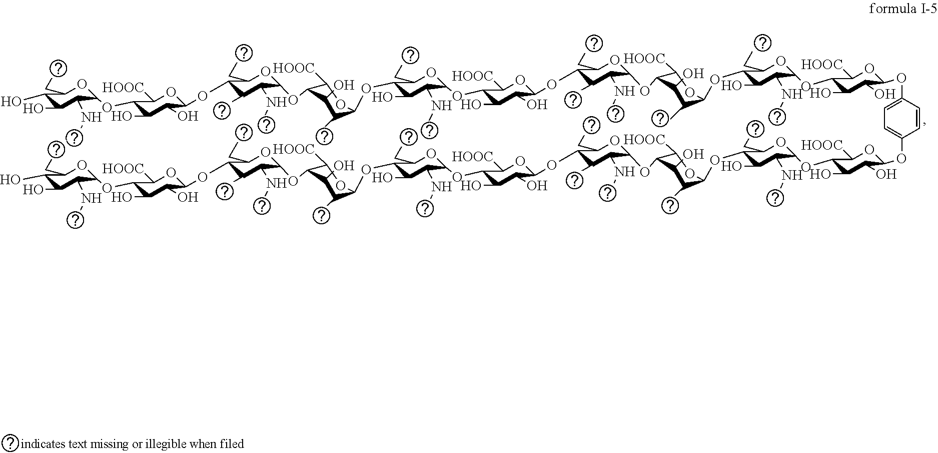
in the formula 1-1 to the formula 1-5, R₁ is the sulfonyl (—SO₃H) or the acetyl (—COCH₃).

5. A method for preparing the anticoagulant phenyl-linked heparin oligosaccharide dimer according to claim 4, comprising combining steps a and b with four or five steps of steps c, d, e, f, and g, wherein the steps a and b are directed to glycosyltransferase catalytic reactions and are conducted at least twice, and the steps c, d, e, f, and g are directed to chemoenzymatic modification reactions as follows with a chemically synthesized dimer intermediate comprising GlcA as a starting material;
the step a, generating a saccharide chain-extended dimeric intermediate by transferring a GlcNTFA residue or a GcNAc residue from UDP-GlcNTFA or UDP-GlcNAc as a glycosyl donor to two non-reducing terminal GlcA residues of either a phenyl bis-O-glucuronide substrate or an oligosaccharide intermediate dimer having GlcA residues at non-reducing ends via α-1,4 glycosidic linkage catalyzed by N-acetylaminoglucosyltransferase (KfiA) or heparosan synthase 2 (PmHS2);
the step b, generating a saccharide chain-extended intermediate by linking a GlcA residue from UDP-GlcA as the glycosyl donor to either glucosamine GlcNTFA or GlcNAc at non-reducing ends of a substrate via β-1,4 glycosidic linkage catalyzed by the PmHS2;
the step c, dissolving the saccharide chain-extended intermediate in an alkaline aqueous solution, letting the alkaline aqueous solution to stand on ice to allow a conversion of all the GlcNTFA residue in a saccharide chain into GlcNH₂ residues through a cleavage of trifluoroacetylate (TFA), converting the GlcNH₂ residues to GlcNS residues under a catalysis of N-sulfotransferase (NST) to obtain an N-sulfated heparin oligosaccharide dimeric intermediate;
the step d, converting all the GlcA residues in a saccharide chain of the N-sulfated heparin oligosaccharide dimeric intermediate located between two GlcNS residues into 2-O-sulfated iduronic acid (IdoA2S) under a co-catalysis of C₅-epimerase (C₅-epi) and 2-O-sulfotransferase (2OST) to obtain an IdoA2S-containing heparin oligosaccharide dimeric intermediate;
the step e, converting all the GlcA residues in a substrate located between the two GlcNS residues into 2-O-sulfated glucuronic acid (GlcA2S) under a catalysis of the 2OST alone;
the step f, generating a 6-O-sulfated heparin-containing oligo dimeric intermediate by sulfating 6-OHs of all the GlcNS residues or the GcNAc residue of the saccharide chain under a co-catalysis of 6-O-sulfotransferase 1 (6OST1) and 6-O-sulfotransferase 3 (6OST3); and
the step g, subjecting 3-OH of GlcNS6S between the GlcA residues and the IdoA2S in a substrate of the 6-O-sulfated heparin-containing oligo dimeric intermediate to a sulfation (GlcNS6S3S) under a catalysis of 3-O-sulfotransferase 1 (3OST1) to obtain the anticoagulant phenyl-linked heparin oligosaccharide dimer as a target compound.

6. The method according to claim 5, wherein the chemically synthesized dimer intermediate comprising the GlcA is phenyl bis-O-glucuronide or substituted phenyl bis-O-glucuronide, aromatic heterocyclic bis-O-glucuronide, substituted aromatic heterocyclic bis-O-glucuronide, C₁-C₅ alkyl bis-O-glucoside, or cyclohexyl bis-O-glucuronide;
the phenyl bis-O-glucuronide as the starting material is prepared by the following process:
taking a fully acetylated glucose as the starting material, removing a terminal acetyl using acetohydrazide to obtain an anomeric hydroxyl-containing intermediate, reacting an exposed hydroxyl at an anomeric position with trichloroacetonitrile (CNCCl₃) to obtain a glycosyl trichloroacetimidate ester, then subjecting the glycosyl trichloroacetimidate ester to a glycosidation with para-diphenol under a participation of an accelerant to produce a bis-O-acetylated glucoside intermediate, and removing acetyls of the bis-O-acetylated glucoside intermediate to obtain phenyl bis-O-glucoside, and finally, selectively co-oxidating C₆—OH by PhI(OAc)₂ and Tempo to yield a target product phenyl bis-O-glucuronoside.

7. The method according to claim 6, wherein a solvent for a reaction of removing the terminal acetyl from the fully acetylated glucose by the acetohydrazide is anhydrous N,N-dimethylformamide (DMF) and the reaction is carried out at a room temperature for 1 hour;
a reaction of the exposed hydroxyl at the anomeric position of the anomeric hydroxyl-containing intermediate with the trichloroacetonitrile (CNCCl₃) is carried out at less than 0° C. for more than 3 hours, with a solvent being a mixed solution of 1,8-diazabicyclo[5.4.0]undec-7-ene (DBU) and anhydrous dichloromethane (DCM);
a reaction of the glycosyl trichloroacetimidate ester with the para-diphenol is performed at −5° C. for 1 hour using anhydrous DCM comprising a molecular sieve as a solvent and BF₃-Et₂O as the accelerant, a resulting reaction solution is extracted by ethyl acetate (EA), dried in vacuum and recrystallized from methanol to obtain the bis-O-acetylated glucoside intermediate; the bis-O-acetylated glucoside intermediate is treated with anhydrous MeOH— sodium methoxide at the room temperature for more than 1 hour to remove the acetyls of the bis-O-acetylated glucoside intermediate, neutralized with an acidic resin to neutrality, filtered, dried in vacuum and crystallized to obtain the phenyl bis-O-glucoside;
the C₆—OH of the phenyl bis-O-glucoside is selectively co-oxidized by the PhI(OAc)₂ and the Tempo, with a solvent used being a mixed solution of t-BuOH, DCM, and H₂O in a volume ratio of 4:4:1, a co-oxidation is conducted overnight at the room temperature and recrystallized from acetonitrile to obtain the target product phenyl bis-O-glucuronoside.

8. The method according to claim 7, wherein the target compound is prepared by adopting a method consisted of the step a, the step b, the step c, the step d, the step f, and the step g using the target product phenyl bis-O-glucuronoside as a raw material, and a synthetic route is shown in Formula III:

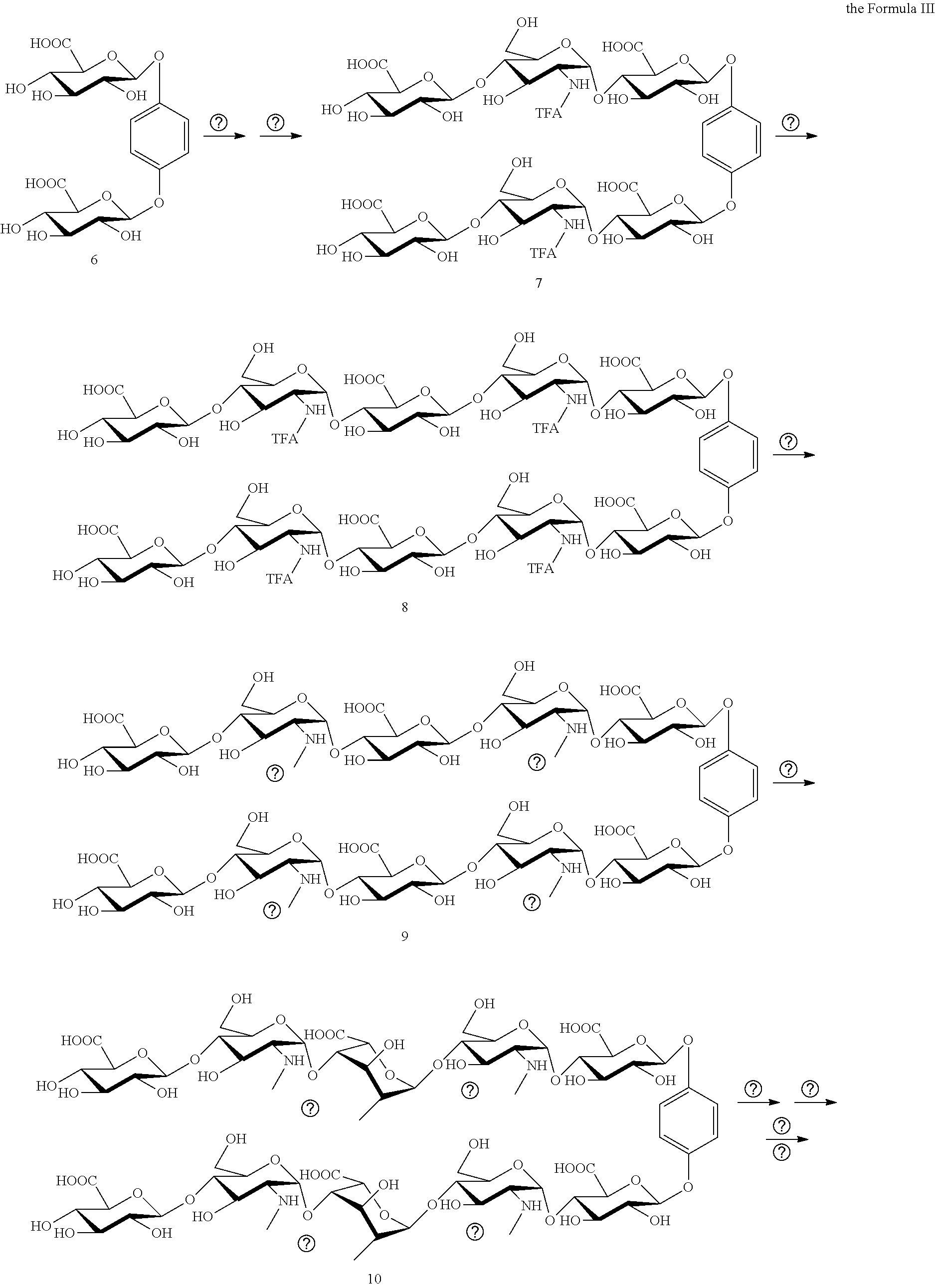

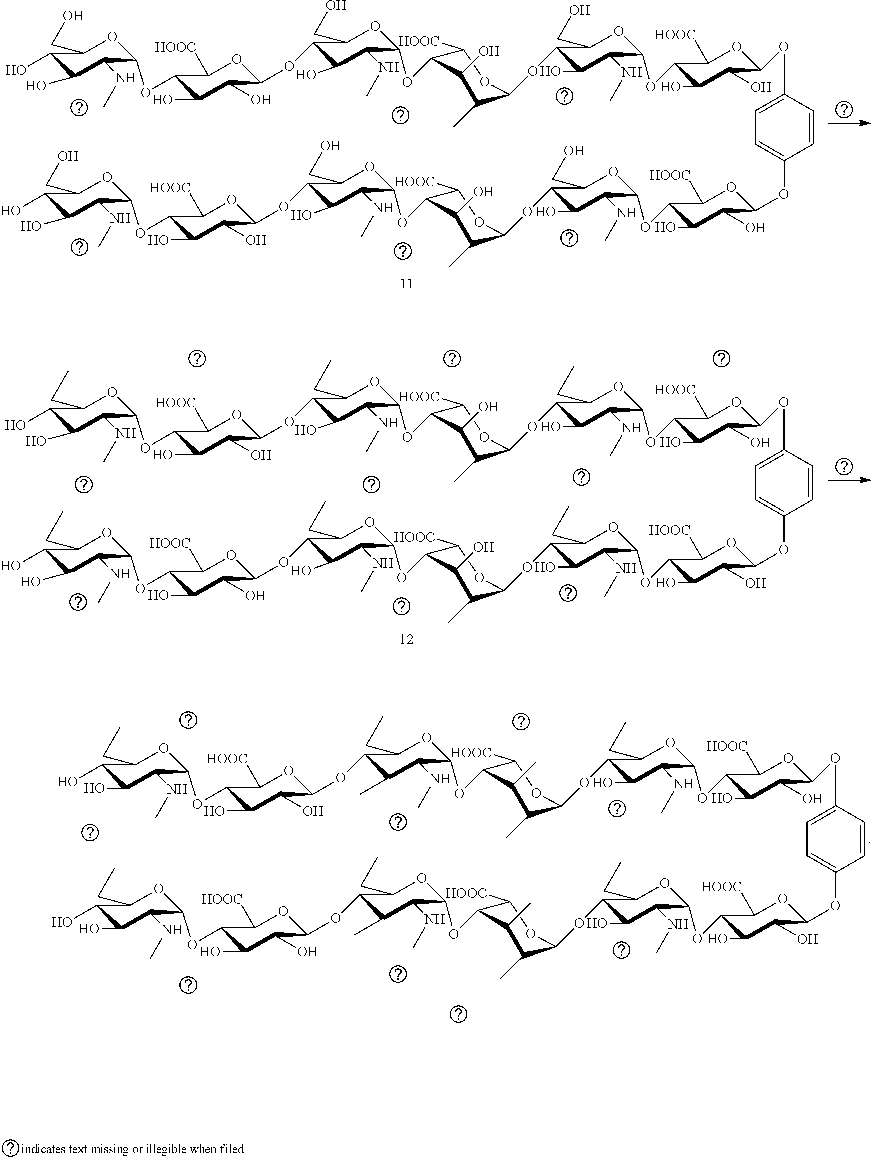

9. The method according to claim 5, wherein synthetic routes for compounds of the formula I-2 to the formula I-5 are as follows:
the formula I-2: the step a→the step b→the step a→the step b→the step c→the step d→the step a→the step b→the step c→the step d→the step a→optional the stet c→the step f→the step g;
the formula I-3: the step a→the step b→the step a→the step b→the step c→the step e→the step a→the step b→the step c→the step d→the step a→optional the step c→the step f→the step g;
the formula I-4 the step a→the step b→the step a→the step b→the step c→the step d→the step a→the step b→the step c→the step d→the step a→the step b→the step c→the step d→the step a→optional the step c→the step f→the step g; and
the formula I-5: the step a→the step b→the step a→the step b→the step c→the stet d→the step f→the step a→the step b→the step a→the step b→the step c→the step d→the step a→optional the step c→the step f→the step g;

10. The method according to claim 5, wherein the KfiA and the PmHS2 are derived from Escherichia coli K5 and Pasteurella multocida, respectively, expressed by Escherichia coli, and a saccharide chain extension reaction catalyzed by an enzyme is performed at 20° C. to 37° C. using a buffer comprising 50 mmol/L tris(hydroxymethyl)aminomethane hydrochloric acid (Tris-HCl) with a pH value of 7.0 to 7.5, a resulting enzymatic product is purified with a reversed-phase chromatography with a C18 column or an anion exchange chromatography to yield a purified product; heparin-modifying enzymes comprising the NST, the C₅-epi, the 2OST, the 6OST1, the 6OST3, and the 3OST1 are obtained by a recombinant expression in the Escherichia coli, a yeast, or insect cells, and catalytic reactions of the heparin-modifying enzymes comprising the NST, the 2OST, the 6OST1, the 6OST3, and the 3OST1 are conducted at 20° C. to 37° C. using 50 mmol/L 2-(N-morpholino) ethanesulfonic acid (MES) with a pH value of 7.0 to 7.5 as a buffer, and 3′-phosphoadenosine-5′-phosphosulfate (PAPS) as a sulfate group donor, and a reaction solution is purified with the anion exchange chromatography to yield a product.

11. (canceled)
